(19) United States
(12) Patent Application Publication
(10) Pub. No.: US 2011/0152923 A1
(43) Pub. Date: Jun. 23, 2011

(54) INCISION CLOSURE DEVICE

(75) Inventor: William D. Fox, New Richmond, OH (US)

(73) Assignee: Ethicon Endo-Surgery, Inc., Cincinnati, OH (US)

(21) Appl. No.: 12/641,853

(22) Filed: Dec. 18, 2009

(51) Int. Cl. A61B 17/00

(52) U.S. Cl. 606/213

(57) ABSTRACT

A surgical instrument which can be inserted into an incision in a patient's stomach, wherein tissue-engaging members positioned within a lumen can be deployed therefrom and can engage the stomach wall surrounding the incision. Once engaged with the stomach wall, the tissue-engaging members can be retracted back into the instrument in order to pull at least a portion of the stomach wall into the instrument. In various embodiments, a cinching member can be utilized to cinch the stomach wall tissue and, as a result, seal the incision. The cinching member can comprise a loop and a pull member, wherein the loop can be disposed around a distal end of the surgical instrument such that it can be slid off of the distal end and around the tissue. The pull member can then be pulled proximally in order to decrease the size of the loop and cinch the tissue.

### BACKGROUND

i. Field of the Invention

The present invention generally relates to surgical devices.

ii. Description of the Related Art

Traditional, or open, surgical techniques may require a surgeon to make large incisions in a patient's body in order to access a tissue treatment region, or surgical site. In some instances, these large incisions may prolong the recovery time of and/or increase the scarring to the patient. As a result, minimally invasive surgical techniques are becoming more preferred among surgeons and patients owing to the reduced size of the incisions required for various procedures. In some circumstances, minimally invasive surgical techniques may reduce the possibility that the patient will suffer undesirable post-surgical conditions, such as scarring and/or infections, for example. Further, such minimally invasive techniques can allow the patient to recover more rapidly as compared to traditional surgical procedures.

Endoscopy is one minimally invasive surgical technique which allows a surgeon to view and evaluate a surgical site by inserting at least one cannula, or trocar, into the patient's body through a natural opening in the body and/or through a relatively small incision. In use, an endoscope can be inserted into, or through, the trocar so that the surgeon can observe the surgical site. In various embodiments, the endoscope may include a flexible or rigid shaft, a camera and/or other suitable optical device, and a handle portion. In at least one embodiment, the optical device can be located on a first, or distal, end of the shaft and the handle portion can be located on a second, or proximal, end of the shaft. In various embodiments, the endoscope may also be configured to assist a surgeon in taking biopsies, retrieving foreign objects, and introducing surgical instruments into the surgical site.

Laparoscopic surgery is another minimally invasive surgical technique where procedures in the abdominal or pelvic cavities can be performed through small incisions in the patient's body. A key element of laparoscopic surgery is the use of a laparoscope which typically includes a telescopic lens system that can be connected to a video camera. In various embodiments, a laparoscope can further include a fiber optic system connected to a halogen or xenon light source, for example, in order to illuminate the surgical site. In various laparoscopic, and/or endoscopic, surgical procedures, a body cavity of a patient, such as the abdominal cavity, for example, can be insufflated with carbon dioxide gas, for example, in order to create a temporary working space for the surgeon. In such procedures, a cavity wall can be elevated above the organs within the cavity by the carbon dioxide gas. Carbon dioxide gas is usually used for insufflation because it can be easily absorbed and removed by the body.

In at least one minimally invasive surgical procedure, an endoscope and/or laparoscope can be inserted through a natural opening of a patient to allow a surgeon to access a surgical site. Such procedures are generally referred to as Nature Orifice Transluminal Endoscopic Surgery or (NOTES)™ and can be utilized to treat tissue while reducing the number of incisions, and external scars, to a patient's body. In various NOTES procedures, for example, an endoscope can include at least one working channel defined therein which can be used to allow the surgeon to insert a surgical instrument therethrough in order to access the surgical site.

The foregoing discussion is intended only to illustrate various aspects of the related art in the field of the invention at the time, and should not be taken as a disavowal of claim scope.

### FIGURES

Various features of the embodiments described herein are set forth with particularity in the appended claims. The various embodiments, however, both as to organization and methods of operation, together with advantages thereof, may be understood in accordance with the following description taken in conjunction with the accompanying drawings as follows.

FIG. 1 is a perspective view of a surgical instrument in accordance with at least one embodiment.

FIG. 2 is a partial cross-sectional view of a handle portion of the surgical instrument of FIG. 1.

FIG. 3 is a perspective view of a distal end of the surgical instrument of FIG. 1 illustrating a shaft, a shaft aperture, and a plurality of hook members positioned within the shaft aperture in an undeployed configuration.

FIG. 4 is a perspective view of the distal end of FIG. 3 illustrating the plurality of hook members in a partially deployed configuration.

FIG. 5 is a perspective view of the distal end of FIG. 3 illustrating the plurality of hook members in a fully deployed configuration.

FIG. 6 illustrates the distal end of FIG. 3 inserted through an opening or incision in the stomach of a patient and the plurality of hook members in their undeployed configuration.

FIG. 7 illustrates the plurality of hook members in their deployed configuration and engaged with a sidewall of the stomach of FIG. 6.

FIG. 8 illustrates the plurality of hook members retracted back into their undeployed configuration and at least a portion of the stomach sidewall pulled into the shaft aperture.

FIG. 9 illustrates a hook member of FIG. 3.

FIG. 10 illustrates the loop of the suture of FIG. 9 moved distally off of the distal end of the surgical instrument and onto the stomach wall tissue.

FIG. 11 illustrates the loop of the suture of FIG. 9 being closed in order to cinch the stomach wall tissue.

Corresponding reference characters indicate corresponding parts throughout the several views. The exemplifications set out herein illustrate various embodiments of the invention, in one form, and such exemplifications are not to be construed as limiting the scope of the invention in any manner.

### DESCRIPTION

Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” or “an embodiment”, or the like, means that a particular feature, structure, or characteristic described in connection with the embodiment is included in at least one embodiment. Thus, appearances of the phrases “in various embodiments,” “in some embodiments,” “in one embodiment,” or “in an embodiment”, or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

In various circumstances, an incision, an opening, or the like, hereinafter referred to as “incision”, can be created in an organ during a surgical procedure. Various devices are known for closing such an incision, such as surgical staplers and clip appliers, for example. Such devices, which may be suitable for their intended purposes, may not be able to sufficiently seal an incision. In various embodiments, a surgical instrument can comprise a shaft which can be inserted into an incision in an organ, such as a stomach, for example, wherein tissue engaging members positioned within the shaft can be deployed therefrom and can engage the stomach wall surrounding the incision. Once engaged with the stomach wall, the tissue engaging members can be retracted back into the shaft and, as a result, pull at least a portion of the stomach wall into the shaft. In various embodiments, a cinching member can be utilized to cinch the stomach wall and, as a result, seal the incision. In at least one embodiment, the cinching member can comprise a loop and a pull member, wherein the loop can be disposed around a distal end of the surgical instrument such that it can be slid off of the distal end and around the stomach wall positioned within, or adjacent to, the distal end of the shaft. Once the loop has been suitably positioned, the pull member can be pulled proximally in order to decrease the size of the loop and cinch the stomach wall such that the incision can be closed and sealed.

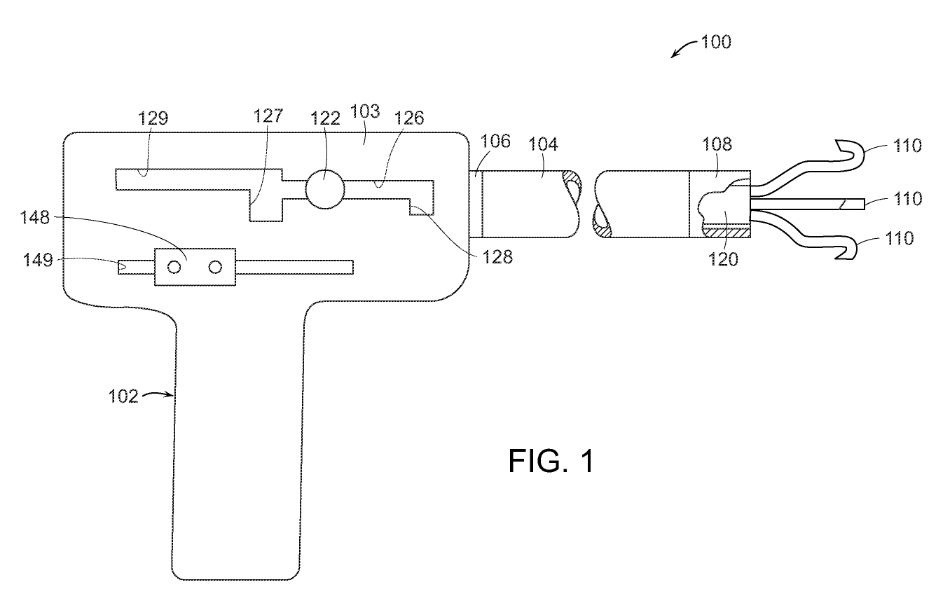

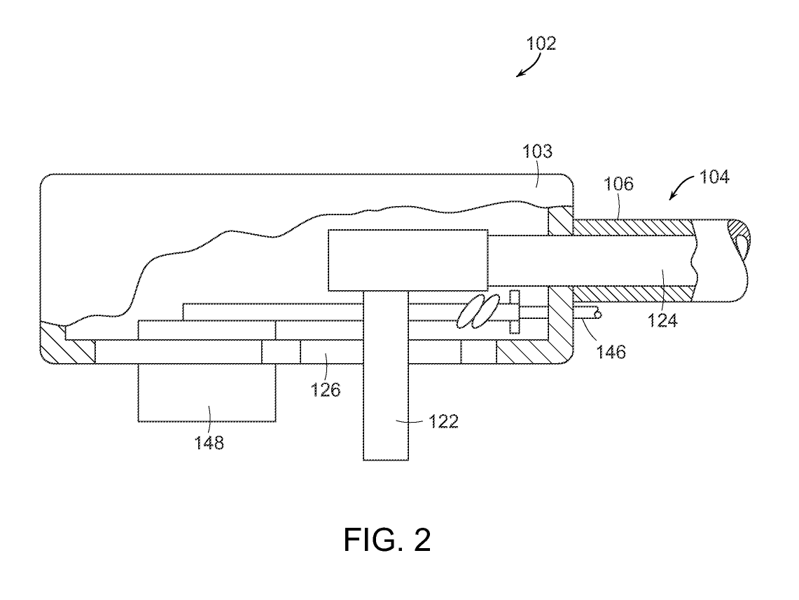

Further to the above, referring now to FIG. 1, a surgical instrument, such as surgical instrument 100, for example, can comprise a handle portion, such as handle portion 102, for example, and a shaft portion, such as elongate shaft portion 104, for example, which can extend from and can be operably engaged with handle portion 102. In various embodiments, shaft portion 104 can comprise a proximal end 106 mounted to handle housing 103 and, in addition, a distal end 108 which can be configured to be inserted into a patient. In at least one such embodiment, referring now to FIG. 2, distal end 108 can be configured to be inserted through an overtube positioned within the esophagus of a patient such that at least the distal end 108 of shaft portion 104 can be positioned within the patient's stomach. In other embodiments, elongate shaft 104 can be configured such that it can be inserted through a patient's esophagus without an overtube. In certain embodiments, distal end 108 of shaft 104 can be inserted into a patient's stomach through an incision in their abdominal wall. In any event, in various embodiments, shaft portion 104 can be comprised of a sufficiently flexible material such that it can be positioned in various curved configurations.

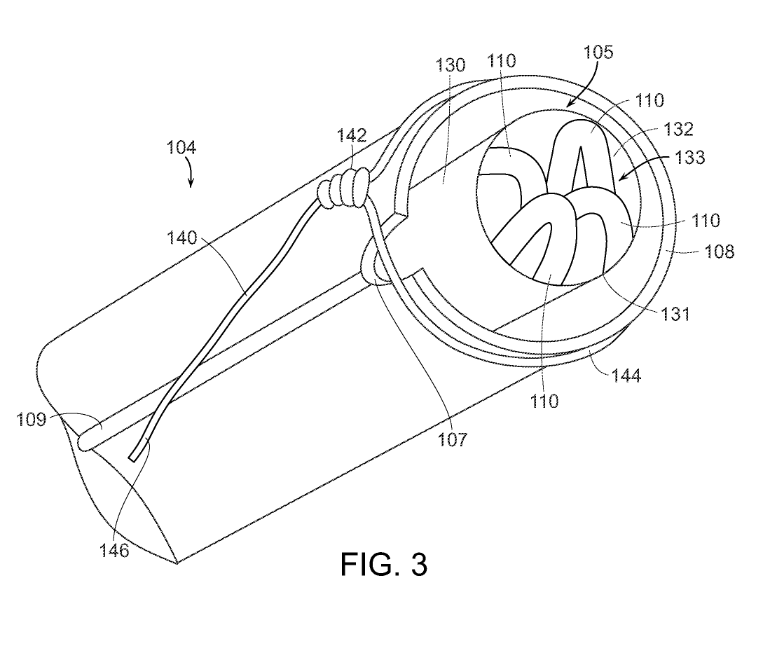

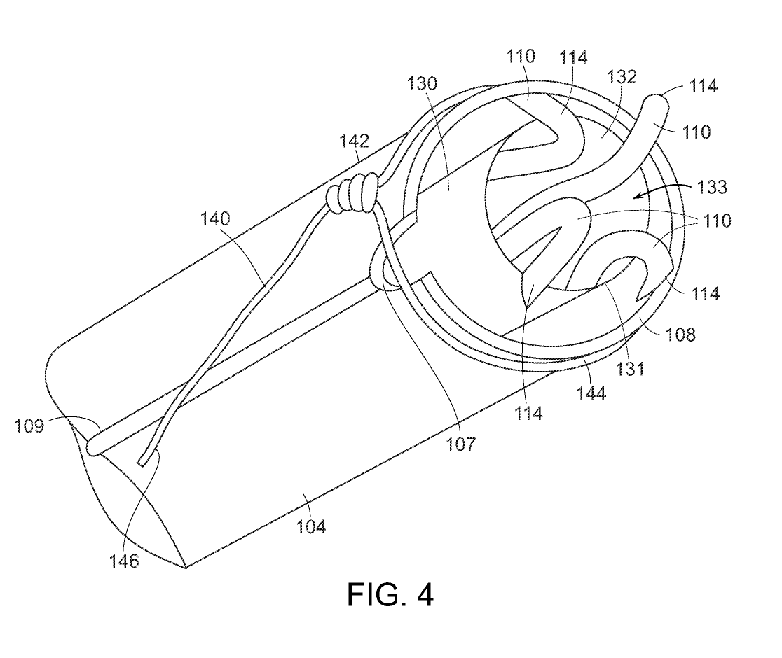

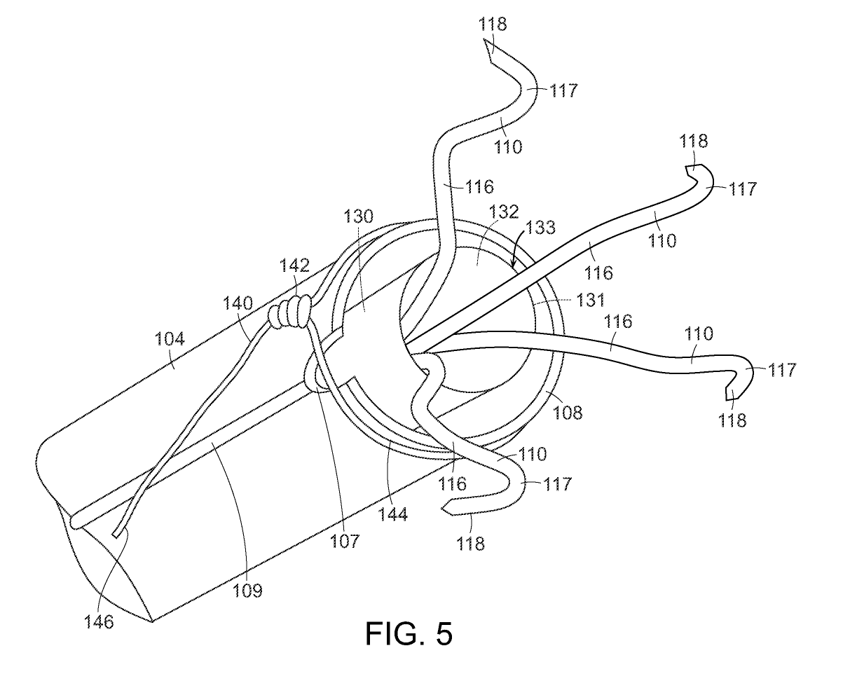

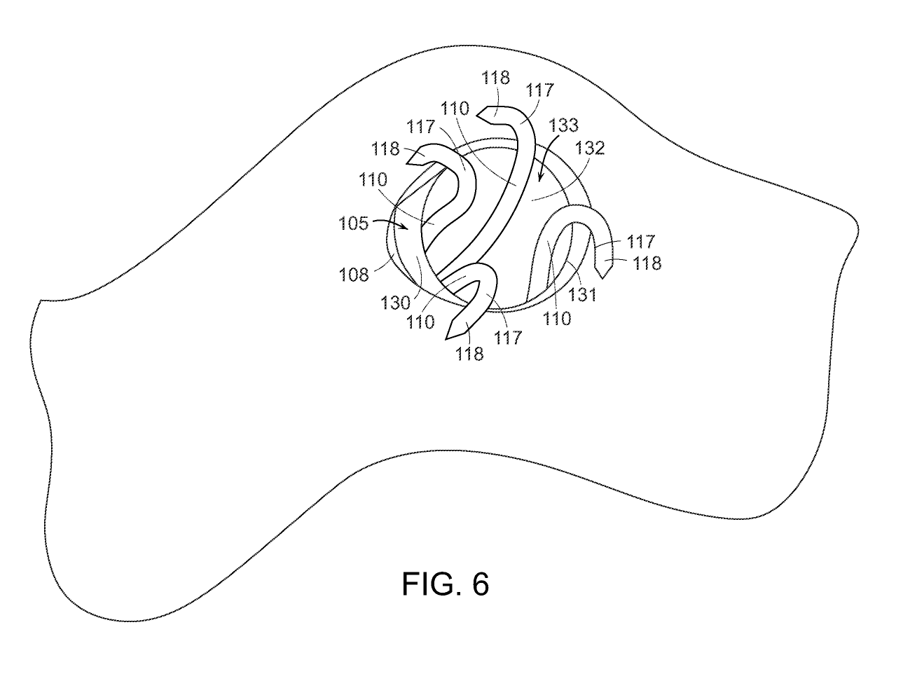

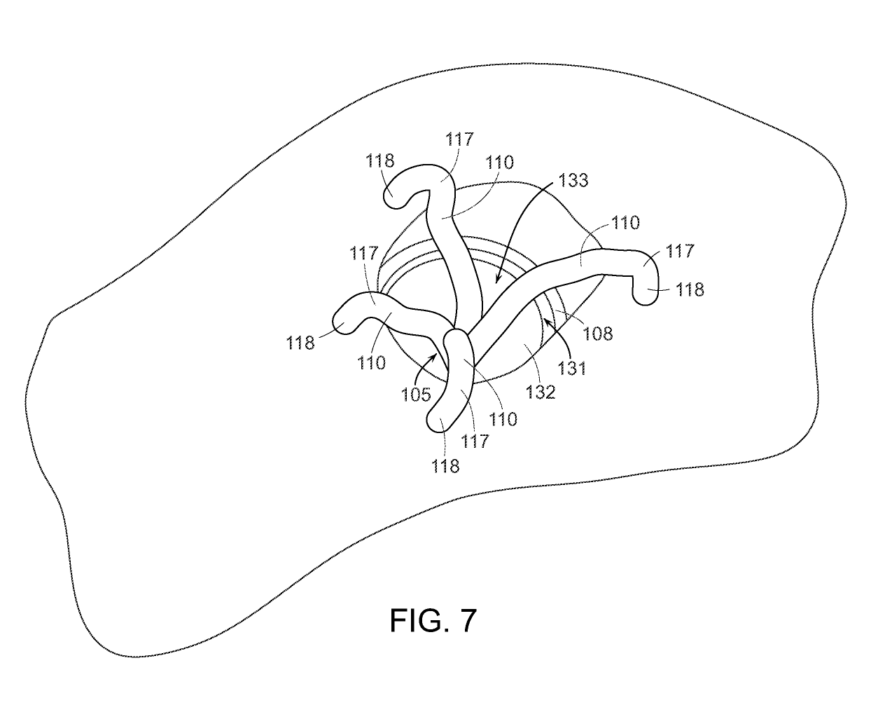

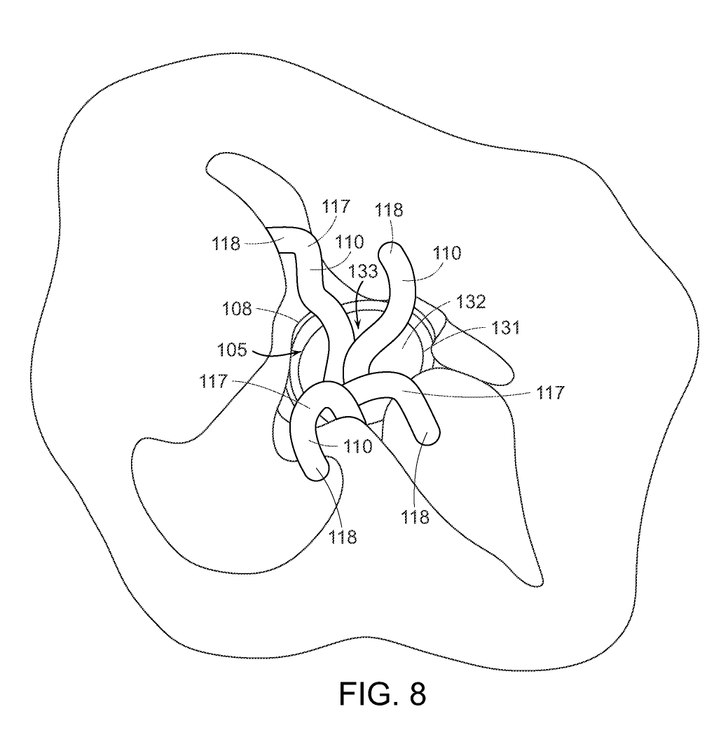

In various embodiments, referring now to FIGS. 3-5, shaft portion 104 can comprise one or more tissue engaging members, such as hook members 110, for example, which can be extended from distal end 108 to engage the sidewalls of a patient's stomach. More particularly, in at least one embodiment, hook members 110 can be moved between an undeployed, or retracted, position, as illustrated in FIG. 3, a partially deployed, or partially extended, position, as illustrated in FIG. 4, and a fully deployed, or fully extended, position, as illustrated in FIG. 5, such that, when hook members 110 are in their fully extended positions, or at least sufficiently extended positions, the hook members 110 can engage the sidewalls of the stomach. In at least one surgical technique, referring now to FIGS. 6-8, the distal end 108 of elongate shaft 104 can be positioned within or adjacent to an incision in a stomach wall, as illustrated in FIG. 6, such that, when the hook members 110 are deployed, the hook members 110 can extend through the incision and engage the outside surface, or lining, of the stomach wall, as illustrated in FIG. 7. Once engaged therewith, referring to FIG. 8, the hook members 110 can be fully retracted, or at least partially retracted, back into shaft member 104. In at least one embodiment, hook members 110 can be sufficiently retracted into shaft aperture 105 such that at least a portion of the stomach sidewall is pulled into, or invaginated within, shaft aperture 105, as described in greater detail further below.

In various embodiments, surgical instrument 100 can comprise four hook members 110, for example. In other various embodiments, a surgical instrument can comprise any other suitable number of hook members 110, such as one, two, three, five, six, seven, eight, nine, and/or ten hook members 110, for example.

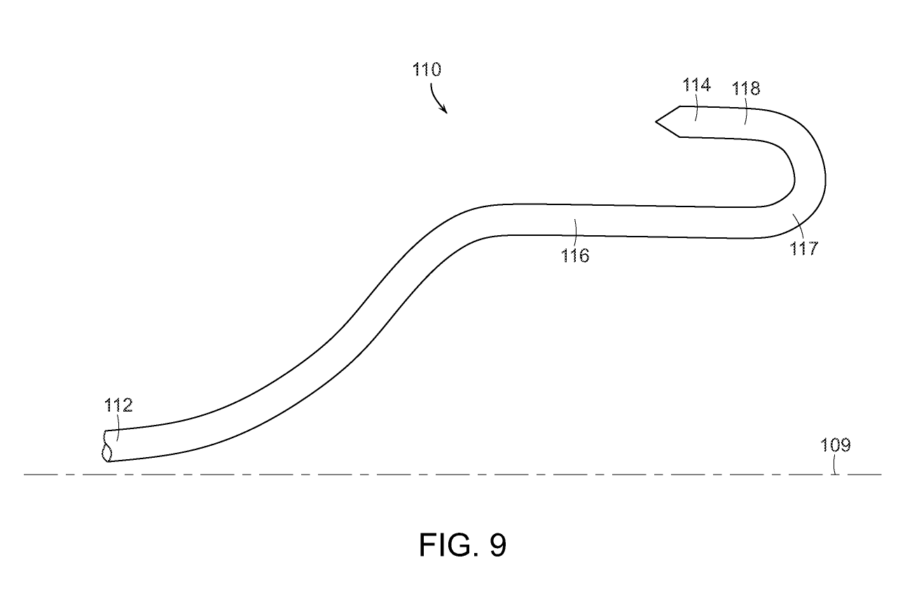

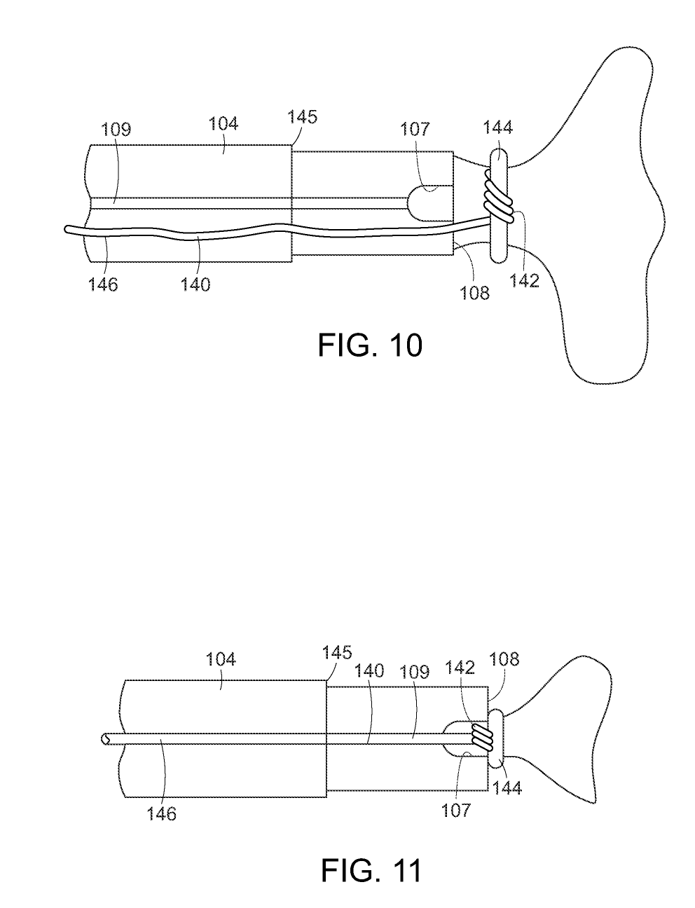

In various embodiments, referring now to FIG. 9, each hook member 110 can comprise a proximal end 112 and a distal end 114, wherein each proximal end 112 can be mounted to at least one slider member, such as slider 120 (FIG. 1), for example. In at least one embodiment, the proximal ends 112 of the hook members 110 can be fixedly mounted to slider 120 such that they do not move relative to slider 120. In at least one such embodiment, slider member 120 can comprise one or more apertures therein, wherein the proximal ends 112 of hook members 110 can be inserted into and/or press-fit within the apertures, for example. In certain embodiments, the proximal ends 112 can comprise one or more points and/or barbs which can be configured to facilitate the insertion of hook members 110 into, and/or facilitate their retention within, slider 120. In various embodiments, although not illustrated, the proximal ends 112 of hook members 110 can be configured to move, or pivot, relative to slider 120. In any event, referring again to FIG. 9, each hook member 110 can further comprise an elongate portion 116, a curved portion 117, and a hook portion 118, for example. In various embodiments, elongate portion 116 can comprise a straight, or an at least substantially straight, configuration which can extend along or be parallel to, or at least substantially parallel to, an axis 109 of shaft 104. Curved portion 117 can extend from elongate portion 116 and can be configured such that a portion thereof extends radially outwardly with respect to axis 109. Each hook portion 118 can extend from a curved portion 117 and can be configured such that they extend in a parallel, or at least substantially parallel, direction with respect to elongate portion 116 and/or axis 109. Alternatively, hooks 108 can extend radially outwardly with respect to axis 109. In various embodiments, further to the above, each hook member 110 can comprise a cantilever, wherein the proximal end 112 of each hook member 110 can be fixed, or at least substantially fixed, to the slider 120, for example, and wherein the distal end 114 of each hook member 110 can be unaffixed to slider 120 and shaft 104 and can move relative to proximal end 112.

In various embodiments, further to the above, the slider 120 can be moved between a first, or proximal, position in which hook members 110 are in an undeployed configuration and a second, or distal, position in which hook members 110 are in a deployed configuration. In at least one embodiment, the surgical instrument 100 can further comprise an anvil or sheath, such as anvil 130 positioned within shaft 104, for example, wherein the anvil 130 can be configured to bias hook members 110 into a radially inward position relative to axis 109. When slider 120 is moved from its proximal position to its distal position, in at least one such embodiment, the distal ends 114 of hook members 110 can at least partially exit anvil 130 and, owing to the elasticity of the material comprising hook members 110, the hook members 110 can splay outwardly relative to axis 109. In at least one embodiment, referring again to FIGS. 3-5 and 9, the hook members 110 can be positioned within anvil 130 such that their distal ends 114 do not extend beyond the distal end 108 of shaft 104 when slider 120 is positioned in its proximal-most position. In such circumstances, the hook members 110 can be deflected, or positioned, inwardly toward axis 109 by anvil 130 such that they remain in their innermost position. As slider 120 is slid from its proximal-most position to its distal-most position, the distal ends 114 of hook members 110 can emerge from the shaft 104 and can begin to splay radially outwardly relative to axis 109. As slider 120 is moved into its distal-most position, the distal ends 114 of hook members 110 can move into their outermost positions. In at least one embodiment, the shaft 104 can comprise a stop positioned within shaft aperture 105 which can limit the travel of slider 120 in the distal direction. In any event, referring again to FIGS. 1 and 2, the handle portion 102 can comprise a lever 122, and/or any other suitable actuator or control, for example, which can be operably coupled to slider 120. In at least one embodiment, lever 122 can be operably coupled to slider 120 via a slider shaft 124, for example. In at least one such embodiment, the lever 122 can be slid distally in order to move slider 120 distally toward distal end 108 of shaft 104 while, correspondingly, the lever 122 can be pulled proximally in order to move slider 120 proximally away from distal end 108 of shaft 104. In certain embodiments, the handle housing 103 can comprise a guide, such as elongate slot 126, for example, which can define a path for lever 122 to move therein.

In various embodiments, the guide, or elongate slot 126, can comprise one or more notches, grooves, detents, and/or recesses which can be configured to receive lever 122 and retain it in position. In at least one embodiment, referring again to FIG. 2, the guide can further include a proximal notch 127 which can be configured to retain lever 122 in a proximal position and a distal notch 128 which can be configured to retain lever 122 in a distal position. In certain embodiments, the guide can further comprise a distal elongate slot 129 which can allow the hook members 110 to be drawn deeper into shaft 104.

In various embodiments, referring again to FIGS. 3-5, the anvil 130 can comprise an inner anvil surface 132 which can define an inner aperture 133, wherein the hook members 110 can contact anvil surface 132 when they are positioned within anvil 130. In certain embodiments, the anvil 130 can comprise an annular, or at least substantially annular, collar and the anvil surface 132 can define a circular, or an at least substantially circular, inner perimeter. In at least one embodiment, the inner and/or outer perimeter of anvil 130 can be continuous and may not have any apertures, slots, and/or reliefs therein. In other embodiments, anvil 130 can comprise one or more apertures, slots, and/or reliefs therein which can be configured to allow anvil 130 to at least partially expand when hook members 110 are positioned therein. In various embodiments, the anvil 130 can comprise a distal end 131 which can be aligned, or at least substantially aligned, with the distal end 108 of shaft 104. In at least one such embodiment, the distal ends 114 of hook members 110 can emerge from anvil 130 and shaft 104 at the same time, or at least substantially the same time. In various other embodiments, the distal end 132 of anvil 130 can be recessed proximally relative to the distal end 108 of shaft 104. In at least one such embodiment, the distal ends 114 of hook members 110 can emerge from anvil 130 before they emerge from shaft 104. In other embodiments, the distal end 132 of anvil 130 can protrude distally from the distal end 108 of shaft 104 such that the distal ends 114 of hook members 110 are not positioned within shaft 104 when they emerge from anvil 130. In any event, in various embodiments, the anvil 130 can be mounted to shaft 104 such that it does not move relative thereto. In certain other embodiments, an anvil can be slidable, extendable and/or retractable relative to shaft 104. In at least one such embodiment, the anvil can move distally as slider 120 is moved distally such that the hook members 110 can remain contained within the anvil as it is moved distally, wherein, after a predetermined amount of travel, the anvil can contact a stop and the hook members 110 can emerge from the anvil and deploy radially outwardly, as described above.

In various embodiments, as described above, the distal end 108 of shaft 104 can be inserted into a patient's stomach, for example, such that the hook members 110 can be deployed through an incision in the stomach wall and engage the stomach wall from the outside. Referring again to FIG. 9, further to the above, the hook portions 110 can extend in a direction which extends both proximally with respect to distal end 108 and radially outwardly with respect to axis 109. In at least one such embodiment, as a result, the hook members 110 can be configured to pull the stomach wall surrounding the incision inwardly into the stomach and into shaft aperture 105 when hook portions 110 are retracted. More particularly, in various embodiments, the lever 122 can be pulled proximally in order to at least partially retract hook members 110 into anvil 130. As hook members 110 are being pulled proximally, in at least one embodiment, the hook members 110 can contact anvil 130 and can be cammed, or biased, radially inwardly toward one another and/or toward axis 109. In various circumstances, the stomach wall engaged by the hook members 110 may at least partially enter into the shaft aperture 105 while, in other circumstances, the stomach wall may not enter into the shaft aperture 105 but may nonetheless be positioned adjacent to distal end 108 of shaft 104. In various embodiments, the distal ends 114 of hook members 110, which can be pointed in a substantially proximal, or backwards-facing, direction, can facilitate the capture and control of the stomach wall tissue.

After the stomach wall tissue has been suitably positioned within and/or relative to the distal end 108 of shaft 104, the stomach wall tissue can be cinched in order to close the incision in the stomach wall. In various embodiments, referring now to FIGS. 11 and 12, surgical instrument 100 can further comprise a suture, such as suture 140, for example, which can be utilized to cinch the stomach wall tissue. In at least one embodiment, suture 140 can comprise an elongate thread, for example, having a first end and a second end, wherein the second end can be tied to create a noose knot, for example. More particularly, the second end of the suture can be tied in a knot 142 in order to create a loop 144, wherein the size of loop 144 can be decreased when the first end of the suture is pulled relative to knot 142. In various embodiments, a noose knot can be created when the second end of the suture is wrapped around another part of the suture one or more times such that the second end can be inserted underneath the wraps and then secured thereunder when the slack is taken out of the wraps. In at least one embodiment, three or more wraps can be used. Such a knot can create a loop in the suture and can allow the loop to be decreased in size, although the knot can also prevent, or at least substantially prevent, the loop from being increased in size. As a result of the above, the perimeter defined by the loop can be decreased after the stomach wall tissue has been positioned within the loop such that the loop can cinch the tissue as the loop is being tightened, or decreased in size. Although a noose knot can be used as described above, any other suitable knot can be used.

In various embodiments, referring again to FIGS. 11 and 12, the loop 144 of suture 140 can be positioned around the distal end 108 of shaft 104 before shaft 104 is inserted into the surgical site. In at least one such embodiment, the distal end 108 can comprise a seat, groove, and/or lip 145 which can be configured to retain, or at least assist in retaining, loop 144 on distal end 108. In use, once the stomach wall tissue has been positioned within and/or relative to the distal end 108, the loop 144 can be slid off of distal end 108 and onto the tissue. In various circumstances, the loop 144 can be slid off of the end of shaft 104 by a grasper, and/or any other suitable surgical instrument. In certain embodiments, the surgical instrument 100 can further comprise a push rod, for example, configured to slide the loop 144 off the end of shaft 104. In any event, in at least one embodiment, the loop 144 can encompass or surround the stomach wall tissue located adjacent to the distal end 108 of the shaft 104. In certain circumstances, the distal end 108 of shaft 104 can be pulled or retracted within the stomach such that the hook members 110 can stretch the stomach wall tissue until the tissue becomes narrower than the outside diameter of the distal end 108. In such circumstances, the loop 144, the diameter of which can be larger than the outside diameter of distal end 108, can be more easily passed onto and around the tissue. Once the loop 144 has been suitably positioned onto and around the stomach wall tissue, the first end, or pull string portion 146, of suture 140 can be pulled proximally in order to reduce the diameter, or perimeter, of loop 144. In various embodiments, a surgeon can pull string portion 146 with their hand. In certain embodiments, the pull string portion 146 can be operably engaged with an actuator, such as slide 148, for example, on handle assembly 102 such that slide 148 can be moved proximally in order to apply a pulling force to pull string portion 146. In at least one embodiment, handle housing 103 can comprise a guide, such as elongate slot 149, for example, which can define a path for slide 148 to move therein.

In various embodiments, the distal end 108 of shaft 104 can comprise one or more recesses, slots, and/or grooves, such as recess 107, for example, which can be configured to receive the knot 142 of suture 140. In at least one such embodiment, the knot 142 can be caught or captured within the recess 107 such that, when a proximal force is applied to suture 140, knot 142 can be prevented or at least inhibited from moving proximally. In such circumstances, the diameter or perimeter of loop 144 can be easily adjusted, for example. In certain embodiments, the shaft 104 can further comprise one or more elongate recesses, slots, and/or grooves, such as groove 109, for example, extending along the length of shaft 104 which can be configured to receive at least a portion of pull string 146.

Once the stomach wall tissue has been suitably cinched, the suture 140 can be cut. In certain embodiments, the suture 140 can be configured to break when a certain magnitude of force is applied thereto. In various embodiments, the hook members 110 can be disengaged from the tissue when a sufficient force is applied thereto. In at least one embodiment, referring to FIG. 13, the lever 122 can be pulled proximally in order to force the distal end 114 of hook members through the stomach wall tissue engaged therewith. In various circumstances, as a result, the distal ends 114 may tear through the tissue positioned within the shaft aperture 105. In such circumstances, however, the tissue being torn would be located inside of the suture loop 144 and, as a result, would be located on the same side as the previously incised tissue and would not disturb the newly created seal, for example. In certain embodiments, the surgical instrument 100 can further comprise a pusher bar, for example, which can be configured to dislodge the stomach wall tissue from the hook members 110. In at least one embodiment, although not illustrated, the pusher bar can be slid around the perimeter of and/or in-between the hook members 110 such that, when the pusher bar contacts the stomach wall tissue, the pusher bar can move the stomach wall tissue distally relative to hook members 110. In any event, once the surgical instrument 100 has been disengaged from the stomach wall tissue, in various embodiments, the shaft 104 can be withdrawn from the patient's stomach through their mouth and esophagus.

In various embodiments, further to the above, a surgical instrument can comprise two or more sutures which can be deployed in order to seal an incision in tissue, for example. More particularly, a surgical instrument can comprise a first suture 140 which can be deployed to create a first, or inner, seal and, in addition, a second suture 140 which can be deployed to create a second, or outer, seal. In various circumstances, such sutures can co-operate to create a more leak-proof seal as compared to a seal created by only one of the sutures. In at least one embodiment, a handle of the surgical instrument can comprise a first actuator for tightening the loop of the first suture and a second actuator for tightening the loop of the second suture.

The embodiments of the devices described herein may be introduced inside a patient using minimally invasive or open surgical techniques. In some instances it may be advantageous to introduce the devices inside the patient using a combination of minimally invasive and open surgical techniques. Minimally invasive techniques may provide more accurate and effective access to the treatment region for diagnostic and treatment procedures. To reach internal treatment regions within the patient, the devices described herein may be inserted through natural openings of the body such as the mouth, anus, and/or vagina, for example. Minimally invasive procedures performed by the introduction of various medical devices into the patient through a natural opening of the patient are known in the art as NOTES™ procedures. Some portions of the devices may be introduced to the tissue treatment region percutaneously or through small—keyhole—incisions.

Endoscopic minimally invasive surgical and diagnostic medical procedures are used to evaluate and treat internal organs by inserting a small tube into the body. The endoscope may have a rigid or a flexible tube. A flexible endoscope may be introduced either through a natural body opening (e.g., mouth, anus, and/or vagina) or via a trocar through a relatively small—keyhole—incision incisions (usually 0.5-1.5 cm). The endoscope can be used to observe surface conditions of internal organs, including abnormal or diseased tissue such as lesions and other surface conditions and capture images for visual inspection and photography. The endoscope may be adapted and configured with working channels for introducing medical instruments to the treatment region for taking biopsies, retrieving foreign objects, and/or performing surgical procedures.

Preferably, the various embodiments of the devices described herein will be processed before surgery. First, a new or used instrument is obtained and if necessary cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility. Other sterilization techniques can be done by any number of ways known to those skilled in the art including beta or gamma radiation, ethylene oxide, and/or steam.

Although the various embodiments of the devices have been described herein in connection with certain disclosed embodiments, many modifications and variations to those embodiments may be implemented. For example, different types of end effectors may be employed. Also, where materials are disclosed for certain components, other materials may be used. The foregoing description and following claims are intended to cover all such modification and variations.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A surgical instrument, comprising:
a handle, comprising;
a first actuator; and
a second actuator;
a shaft, comprising:
a proximal end coupled to said handle; and
a distal end configured to be inserted into a patient;
a first hook member configured to engage tissue, wherein said first hook member is movable between an undeployed position and a deployed position, wherein said first actuator is operably coupled with said first hook member to move said first hook member between its undeployed position and its deployed position;
a second hook member configured to engage tissue, wherein said second hook member movable between an undeployed position and a deployed position, wherein said first actuator is operably coupled with said second hook member to move said second hook member between its undeployed position and its deployed position, wherein at least a portion of said first hook member and at least a portion of said second hook member are positionable within said shaft when said first hook member and said second hook member are in their undeployed positions, and wherein at least a portion of said first hook member and at least a portion of said second hook member extend distally from said distal end of said shaft when said first hook member and said second hook member are in their deployed positions; and
a cinching assembly, comprising:
a pull member; and
a loop, wherein said cinching assembly is slidable between a proximal position and a distal position, wherein said loop is positioned around said distal end of said shaft when said cinching assembly is in said proximal position, wherein said loop is positioned distally relative to said distal end of said shaft when said cinching assembly is in its distal position, wherein said pull member is operably coupled with said second actuator, and wherein said second actuator is configured to pull said pull member proximally and at least partially close said loop.

2. The surgical instrument of claim 1, wherein said shaft defines an axis, wherein said first hook member comprises an end which extends away from said axis, and wherein said second hook member comprises an end which extends away from said axis.

3. The surgical instrument of claim 1, wherein said shaft defines an axis, wherein said first actuator comprises a control slidable between a first position and a second position, wherein said control is slidable from said first position into said second position to move said first hook member and said second hook member into their deployed positions, and wherein said control is slidable from said second position into said first position to move said first hook member and said second hook member into their undeployed positions.

4. The surgical instrument of claim 1, further comprising an anvil sheath, wherein said anvil sheath is configured to bias said first hook member toward said second hook member when said first hook member and said second hook member are in their undeployed positions.

5. The surgical instrument of claim 4, wherein said anvil sheath is configured to bias said second hook member toward said first hook member when said first hook member and said second hook member are in their undeployed positions, and wherein said first hook member and said second hook member are configured to splay outwardly with respect to each other when said first hook member and said second hook member are moved into their deployed positions.

6. The surgical instrument of claim 1, further comprising a slider member positioned within said shaft, wherein said first actuator is operably engaged with said slider member, wherein said first hook member comprises a first cantilever having an end engaged with said slider member, and wherein said second hook member comprises a second cantilever having an end engaged with said slider member.

7. The surgical instrument of claim 1, wherein said pull member and said loop are comprised of a suture, wherein said suture includes a knot to form said loop, and wherein said second actuator is configured to pull said pull member relative to said knot to decrease the size of said loop.

8. A surgical instrument, comprising:
a handle comprising an actuator;
a shaft, comprising:
a shaft aperture;
a proximal end coupled to said handle; and
a distal end configured to be inserted into a patient;
a first hook member configured to engage tissue of the patient, wherein said first hook member is movable between a retracted position and an extended position;
a second hook member configured to engage tissue of the patient, wherein said second hook member is movable between a retracted position and an extended position, wherein at least a portion of said first hook member and at least a portion of said second hook member are positionable within said shaft aperture when said first hook member and said second hook member are in their retracted positions, wherein at least a portion of said first hook member and at least a portion of said second hook member extend distally from said distal end of said shaft when said first hook member and said second hook member are in their extended positions, wherein said first hook member and said second hook member are operably engaged with said actuator, and wherein said actuator is configured to move said first hook member between its retracted position and its extended position and said second hook member between its retracted position and its extended position; and
an anvil sheath configured to resiliently move said first hook member toward said second hook member when said first hook member is moved into its retracted position, and wherein said first hook member is configured to move away from said second hook member when said first hook member is moved into its extended position.

9. The surgical instrument of claim 8, wherein said shaft defines an axis, wherein said first hook member comprises an end which extends away from said axis, and wherein said second hook member comprises an end which extends away from said axis.

10. The surgical instrument of claim 8, wherein said shaft defines an axis, wherein said actuator comprises a control slidable between a first position and a second position, wherein said control is slidable from said first position into said second position to move said first hook member and said second hook member into their extended positions, and wherein said control is slidable from said second position into said first position to move said first hook member and said second hook member into their retracted positions.

11. The surgical instrument of claim 8, wherein said anvil sheath is configured to bias said second hook member toward said first hook member when said first hook member and said second hook member are in their retracted positions, and wherein said first hook member and said second hook member are configured to splay outwardly with respect to each other when said first hook member and said second hook member are moved into their extended positions.

12. The surgical instrument of claim 8, further comprising a slider member positioned within said shaft, wherein said actuator is operably engaged with said slider member, wherein said first hook member comprises a first cantilever having an end engaged with said slider member, and wherein said second hook member comprises a second cantilever having an end engaged with said slider member.

13. The surgical instrument of claim 8, further comprising:
a cinching assembly, comprising:
a pull member; and
a loop, wherein said cinching assembly is slidable between a proximal position and a distal position, wherein said loop is positioned around said distal end of said shaft when said cinching assembly is in said proximal position, wherein said loop is positioned distally relative to said distal end of said shaft when said cinching assembly is in its distal position, and wherein said pull member is configured to be pulled proximally to at least partially close said loop.

14. The surgical instrument of claim 13, wherein said pull member and said loop are comprised of a suture, wherein said suture includes a knot to form said loop, and wherein said pull member is configured to be pulled relative to said knot to decrease the size of said loop.

15. A surgical instrument, comprising:
a handle comprising an actuator;
a shaft, comprising:
a proximal end coupled to said handle; and
a distal end configured to be inserted into a patient;
a hook assembly, wherein said actuator is configured to move said hook assembly between a first position and a second position, said hook assembly comprising
a slider operably coupled with said actuator;
a first hook member, comprising:
a first attached end mounted to said slider; and
a first free end configured to engage tissue of the patient, wherein said first free end is movable relative to said first attached end; and
a second hook member, comprising:
a second attached end mounted to said slider; and
a second free end configured to engage tissue of the patient, wherein said second free end is movable relative to said second attached end;
an anvil, wherein said anvil defines an axis, wherein said anvil is configured to position said first hook member and said second hook member relative to said axis when said hook assembly is in said first position, and wherein said first hook member and said second hook member are configured to resiliently splay outwardly away from said axis when said hook assembly is moved from said first position into said second position.

16. The surgical instrument of claim 15, wherein said first free end of said first hook member and said second free end of said second hook member are positioned distally with respect to said distal end of said shaft when said hook assembly is in said second position.

17. The surgical instrument of claim 15, further comprising:
a cinching assembly, comprising:
a pull member; and
a loop, wherein said cinching assembly is slidable between a proximal position and a distal position, wherein said loop is positioned around said distal end of said shaft when said cinching assembly is in said proximal position, wherein said loop is positioned distally relative to said distal end of said shaft when said cinching assembly is in its distal position, and wherein said pull member is configured to be pulled proximally to at least partially close said loop.

18. The surgical instrument of claim 15, wherein said pull member and said loop are comprised of a suture, wherein said suture includes a knot to form said loop, and wherein said pull member is configured to be pulled relative to said knot to decrease the size of said loop.

19. The surgical instrument of claim 15, wherein said anvil comprises an aperture, and wherein at least a portion of said first hook member and at least a portion of said second hook member are configured to be positioned within said aperture when said hook assembly is in said first position.

20. The surgical instrument of claim 19, wherein said aperture is defined by sidewalls, and wherein said first hook member and said second hook member are held in deflected positions by said sidewalls when said hook assembly is in said first position.
